(19) United States
(12) Patent Application Publication
(10) Pub. No.: US 2023/0266320 A1
(43) Pub. Date: Aug. 24, 2023

(54) METHOD FOR DETECTING CYTOMEGALOVIRUS (CMV) AND MEASURING AND QUANTIFYING PENTAMERIC COMPLEX USING AN INDIRECT SANDWICH ELISA

(71) Applicant: MERCK SHARP & DOHME CORP., Rahway, NJ (US)

(72) Inventors: Cindy J. Pauley, Lansdale, PA (US); Lawrence William Dick, Jr., Norristown, PA (US); Mary Lillian Shank Retzlaff, Norristown, PA (US); Matthew C. Troutman, Lansdale, PA (US); Lynne Ireland Denny, Collegeville, PA (US); Qiana M. Wilson, Philadelphia, PA (US); Kuo-Chang Yin, Ambler, PA (US)

(73) Assignee: Merck Sharp & Dohme LLC, Rahway, NJ (US)

(21) Appl. No.: 17/779,011

(22) PCT Filed: Nov. 20, 2020
(86) PCT No.: PCT/US2020/061437

§ 371 Date: May 23, 2022

Related U.S. Application Data
(60) Provisional application No. 62/940,603, filed on Nov. 26, 2019.

(51) Int. Cl. G01N 33/569

(52) U.S. Cl. CPC G01N 33/56994; G01N 2333/045; G01N 2470/04

(57) ABSTRACT

The present invention relates to a method of detecting the presence of Cytomegalovirus and measuring antigenicity through detection and quantification of a pentameric complex by an indirect sandwich ELISA assay which ensures an appropriate concentration of this critical glycoprotein complex is present in the vaccine.

### BACKGROUND OF THE INVENTION

Cytomegalovirus (CMV), also known as human herpes virus 5 (HHV-5), is classified as being a member of the beta subfamily of herpesviridae. According to the Centers for Disease Control and Prevention, CMV infection is found ubiquitously in the human population, with an estimated 40-80% of the United States adult population having been infected. The virus is spread primarily through bodily fluids and is frequently passed from pregnant mothers to the fetus or newborn. In most individuals, CMV infection is latent, although virus activation can result in high fever, chills, fatigue, headaches, nausea, and splenomegaly.

Although most human CMV infections are asymptomatic, CMV infections in immunocompromised individuals, (such as HIV-positive patients, allogeneic transplant patients and cancer patients) or persons whose immune system has yet fully developed (such as newborns) can be particularly problematic (Mocarski et al., Cytomegalovirus, in Field Virology, 2701-2772, Editor: Knipes and Howley, 2007). CMV infection in such individuals can cause severe morbidity, including pneumonia, hepatitis, encephalitis, colitis, uveitis, retinitis, blindness, and neuropathy, among other deleterious conditions.

A significant contribution to the development of a HCMV vaccine was the identification of the pentameric complex (PC), which consists of HCMV gH, gL, pUL128, pUL130, and pUL 131 (Ryckman et al., Characterization of the Human Cytomegalovirus gH/gL/UL128-131 Complex that Mediates entry into Epithelial and Endothelial cells, J. Virol., 2008, 82, 60-70).

CMV infects various cells in vivo, including monocytes, macrophages, dendritic cells, neutrophils, endothelial cells, epithelial cells, fibroblasts, neurons, smooth muscle cells, hepatocytes, and stromal cells (Plachter et al., Cell types involved in replication and distribution of human Cytomegalovirus, Adv Virus Res, 46. 1996, 195-261). Although clinical CMV isolates replicate in a variety of cell types, laboratory strains AD169 (Elek et al., Development of a Vaccine Against Mental Retardation Caused by Cytomegalovirus Infection in Utero, Lancet, 1 (1974), 1-5) and Towne (Plotkin et al., Candidate Cytomegalovirus strain for human vaccination, Infection and Immunity, 12(3), 1975, 521) replicate almost exclusively in fibroblasts (Hahn et al., J. Virol., 2004, 78, 10023-10033). The restriction in tropism, which results from serial passages and eventual adaptation of the virus in fibroblasts, is stipulated as a marker of attenuation (Gerna et al., Dendritic-cell infection by human Cytomegalovirus is restricted to strains carrying functional UL131-128 genes and mediates efficient viral antigen presentation to D8+ T cells, Journal of General Virology, 86, 2005, 275-284; Gerna et al, J. Gen Virol. 83, 2002, 1993; Gerna et al, J. Gen Virol. 84, 2003, 1431; Dargan et al, J. Gen Virol. 91, 2010, 1535). Mutations causing the loss of epithelial cell, endothelial cell, leukocyte, and dendritic cell tropism in human CMV laboratory strains have been mapped to three open reading frames (ORFs): UL128, UL130, and UL131 (Hahn et al., J. Virol., 2004, 78, 10023; Wang and Shenk, J. Virol., 2005, 79, 10330; Wang and Shenk, Proc Natl Acad Sci USA., 2005, 102, 18153). Biochemical and reconstitution studies show that UL128, UL130 and UL131 assemble onto a gH/gL scaffold to form a pentameric gH complex (Wang and Shenk, Proc Natl Acad Sci USA., 2005, 102, 1815; Ryckman et al, J. Virol., 2008, 82:60). Restoration of this complex in virions restores the viral epithelial tropism in the laboratory strains (Wang and Shenk, J. Virol., 2005, 79, 10330).

Loss of endothelial and epithelial tropism has been suspected as a deficiency in the previously evaluated strains such as Towne (Gerna et al, J. Gen Virol., 2005, 83, 1993; Gerna et al, J. Gen Virol., 2003, 84, 1431). Neutralizing antibodies in sera from human subjects of natural CMV infection have more than 15-fold higher activity against viral epithelial entry than against fibroblast entry (Cui et al, Vaccine, 2008, 26, 5760). Humans with primary infection rapidly develop neutralizing antibodies to viral endothelial and epithelial entry but only slowly develop neutralizing antibodies to viral fibroblast entry (Gema et al, J. Gen. Virol., 2008, 89, 853). Furthermore, neutralizing activity against viral epithelial and endothelial entry is absent in the immune sera from human subjects who received Towne vaccine (Cui et al, Vaccine, 2008, 26, 5760). More recently, a panel of human monoclonal antibodies from four donors with HCMV infection was described, and the more potent neutralizing clones from the panel recognized the antigens of the pentameric gH complex (Macagno et al, J. Virol., 2010, 84, 1005).

The early efforts on HCMV vaccine focused on live attenuated viruses, such as AD169 and Towne strains. These attenuated strains were developed by adapting the virus in human fibroblasts such as MRC-5 cells. Attenuated HCMV vaccines are reportedly safe with no detectable peripheral leukocyte viral loads or urine viral shedding in vaccinated subjects, indicating there is no viral replication outside the injection site. The attenuation phenotypes, as well as the poor immunogenicity’s, were believed to be associated with the genetic mutations introduced by extensive propagation in fibroblasts. Recent genetic analysis revealed that Towne and AD169 share common and substantial mutations at the right end of the unique long region (Murph, E et al., Proc Natl Acad Sci USA, 2003, 100, 14976-14981). A 13-15 Kb fragment in the UL/b′ region of viral genome was deleted and replaced by an inverted duplication of unique short region, when compared with limit of quantitation passage viral isolates. The deletion in AD169 contains ORFs UL132 to UL145. This deletion has been reported to be essential for the virus to replicate in human fetal tissues engrafted in SCID mice (Wang and Shenk, J. Virol., 2005, 79, 10330-10338 or Wang and Shenk, Proc Natl Acad Sci USA, 2005, 102, 18153 - 18158). The deletion of UL 138 gene also impaired the ability of the virus to establish latency in hematopoietic cells (Goodrum and Shenk). These large-scale gene rearrangements were accompanied by additional point mutations, such as frame-shifts in genes RL5A, RL13, and UL131 (Sijmons et al., Genomic and Functional Characteristics of Human Cytomegalovirus Revealed by Next-Generation Sequencing, 2014, 6(3), 1049-1072).

A newly discovered gH complex has been linked to viral endothelial and epithelial tropism and this complex is missing in all laboratory strains due to a variety of mutations in viral UL128, UL130, and UL131 locus, adjacent to the UL/b′ deletion (Hahn G. et al, (2004) J Virol 78, 10023-10033). Studies have shown that sequential passages of a clinical strain in fibroblasts led to multiple mutations in viral genomes, including the ones in the UL131-128 locus affecting the recently defined gH complex (Dargen, D.J, et al (2010) J Gen Virol 91, 1535-1546; Stanton, R.J. et al, (2010) J. Clin Invest 120, 3191-3208). The viral epithelial tropism can be restored in the laboratory strains, either by complementation of a DNA fragment containing the UL131-128 locus or genetically repairing a frame-shift mutation in UL131 ORF (Hahn G. et al, (2004) J Virol 78, 10023-10033). These studies demonstrate that the proteins encoded in the UL131-128 locus are necessary and sufficient for restoring viral tropisms to epithelial and endothelial cells in the laboratory strains. Further biochemical and reconstitution studies showed that this gH complex is composed of five proteins, and its configuration and incorporation into mature progeny virions requires a proper assembly of three proteins encoded by UL128, UL130 and UL131 onto gH/gL scaffold. The complex is therefore referred as the pentameric gH complex to distinguish it from the gH/gL/gO complex (Ryckman et al., Characterization of the human Cytomegalovirus gH/gL/UL128-131 complex that mediates entry into epithelial and endothelial cells, (2008) J. Virol 82(1), 60-70; Wang et al., Human Cytomegalovirus UL131 open reading frame is required for epithelial cell tropism, (2005) J. Virol. 79(16), 10330-8).

Poor immunogenicity of live attenuated HCMV vaccines, such as Towne, was first hypothesized as a result of its losing endothelial tropism in 2002 (Gerna, G. et al., J Gen Virol 83, 1993-2000). It was shown later that neutralizing Ab titers, in CMV seropositive subjects, were about 8 to 10-fold higher against viral endothelial and epithelial entry than against fibroblast entry, suggesting the importance of viral epithelial and endothelial entry in natural HCMV infections (Cui, X, (2008) Vaccine, 26, 5760-5766). The neutralizing activity against viral epithelial and endothelial entry is missing in the immune sera from Towne vaccinated subjects, supporting the hypothesis raised in 2002 (Macagno, A et al, (2010), J Virol 84, 1005-1013). More recently, a panel of human mAbs isolated from four seropositive donors was described, and the potent neutralizing mAbs were shown to recognize the antigens or epitopes of the pentameric gH complex (Macagno, A et al, (2010), J Virol 84, 1005-1013). These observations highlight the importance of the pentameric gH complex as an important target of effective neutralizing Abs in human with natural HCMV infection, and more importantly, suggest its role in developing effective HCMV vaccines.

An enzyme-linked immunosorbent assay (ELISA) is a biochemistry assay that uses a solid phase enzyme immunoassay (EIA) to detect the presence of an antigen of interest. In an ELISA, an antigen is captured by an immobilized antibody and then complexed with an antibody that is linked to an enzyme. Detection is accomplished by assessing the conjugated enzyme activity via incubation with a substrate to produce a measurable product. Immobilization of the antigen of interest can be accomplished by direct adsorption to the assay plate or indirectly via a capture antibody that has been attached to the plate. The antigen is then detected either directly (labelled primary antibody) or indirectly (labelled secondary antibody).

The sandwich ELISA consists of the analyte to be measured bound between two primary antibodies – the capture antibody and the detection antibody. A coating antibody is directly attached to the plate, which binds to the capture antibody. The capture antibody is highly specific for the antigen. The antigen is then added, followed by addition of a second antibody referred to as the detection antibody. The detection antibody binds the antigen at a different epitope than the capture antibody. As a result, the antigen is ‘sandwiched’ between the two antibodies. Either monoclonal or polyclonal antibodies can be used as the capture and detection antibodies in sandwich ELISA systems. Monoclonal antibodies have an inherent monospecificity toward a single epitope that allows fine detection and quantitation of small differences in antigen.

### SUMMARY OF THE INVENTION

The present invention relates to a method of detecting the presence of Cytomegalovirus and measuring antigenicity through detection and quantification of a pentameric complex.

The present invention relates to a method of measuring antigenicity through detection of the pentameric complex by an indirect sandwich ELISA assay and quantification of this critical glycoprotein complex as compared to a reference standard. The present invention ensures there is a consistent level of antigenicity or pentameric content per dose.

### BRIEF DESCRIPTION OF THE DRAWINGS

FIG. 1 is a line graph depicting the relative fluorescence units versus concentration comparing a direct and indirect ELISA.

FIG. 2 is a line graph depicting the relative fluorescence units versus concentration for overnight sample incubation versus 2-hour sample incubation.

FIG. 3 is a line graph showing either assay, 57.4C_25.2H or 57.4C_D1-103H (also referred to as 57.4_25.2H or 57.4C_D1-103H), is a suitable pair for measuring the pentameric complex.

FIG. 4 is a line graph depicting the comparison of antigen content results for drug product and drug substance matrices using the Manual ELISA vs. the D300 Digital Dispenser ELISA.

FIG. 5 is a bar chart representing the comparison of different detergents evaluated for use in the ELISA assay.

FIGS. 6A and 6B are particle profiles generated using flow cytometry representing the comparison of different detergents evaluated for use in the ELISA assay. FIG. 6B shows an earlier PS20 particle profile than that of P188 buffer.

### DETAILED DESCRIPTION OF THE INVENTION

The present invention relates to a method for detecting the presence of Cytomegalovirus in a sample, comprising measuring the amount of pentameric complex in a drug substance, drug product, or additional process intermediary samples using an indirect sandwich enzyme linked immunosorbent assay (ELISA).

A subembodiment of the present invention relates to a method for detecting the presence of Cytomegalovirus through the use of two monoclonal antibodies, measuring the amount of the pentameric complex in a sample using an indirect sandwich enzyme linked immunosorbent assay (ELISA).

In a further subembodiment of the present invention, the sample is a drug substance sample, drug product sample, or additional process intermediary samples.

In a further subembodiment of the present invention, is a method for detecting and quantifying the presence of Cytomegalovirus without detecting and quantifying multiple glycoproteins

A subembodiment of the present invention is a method for detecting the presence of Cytomegalovirus in a sample, comprising measuring the amount of the pentameric complex, wherein the pentameric complex comprises gH, gL, UL128, UL130, and UL131, and wherein, two monoclonal antibodies against different epitopes on the pentameric complex are used to detect and quantitate the glycoprotein complex, and the pentameric complex is quantitated through an ELISA against a reference standard with a known amount of CMV.

In a further subembodiment of the present invention, the two monoclonal antibodies comprise an IgG1 mouse-rabbit chimeric antibody and a human antibody isolated from patients with a naturally occurring CMV infection. In a further subembodiment of the present invention, the IgG1 mouse-rabbit chimeric antibody is 57.4C. In a further subembodiment of the present invention, the IgG1 mouse-rabbit chimeric antibody is a capture antibody. In a further subembodiment of the present invention, the human antibody is D1-103H or 25.2. In a further subembodiment of the present invention, the human antibody is a detection antibody.

A subembodiment of the present invention is an indirect sandwich ELISA assay detecting the presence of Cytomegalovirus comprising measuring the amount of the pentameric complex, wherein the pentameric complex comprises gH, gL, UL128, UL130, and UL131, wherein the relative potency against a reference standard is used to calculate the antigen content of the sample.

A subembodiment of the present invention is a method of detecting the presence of Cytomegalovirus (CMV) in a sample, comprising measuring the amount of a pentameric complex in a sample using an indirect sandwich ELISA comprising two monoclonal antibodies, and quantifying the pentameric complex amount against a reference standard with a known amount of CMV by relative potency, wherein the pentameric complex comprises gH, gL, UL128, UL130, and UL131, and wherein the two monoclonal antibodies comprise an IgG1 mouse-rabbit chimeric antibody and a human antibody isolated from patients with a naturally occurring CMV infection.

A subembodiment of the present invention is wherein the IgG1 mouse-rabbit chimeric antibody is 57.4C, and wherein the human antibody is either D1-103 or 25.2.

A subembodiment of the present invention is a diagnostic system for detecting the presence of Cytomegalovirus, said system including an ELISA assay measuring the amount of the pentameric complex.

A subembodiment of the present invention is a method for treating a patient with CMV by administering a vaccine against CMV after detecting the presence of Cytomegalovirus in a sample, comprising measuring the amount of the pentameric complex in a drug substance, drug product, or additional process intermediary samples using an indirect sandwich enzyme linked immunosorbent assay (ELISA). A further subembodiment of the invention is wherein the ELISA measures CMV dose. A further subembodiment of the invention is wherein the ELISA is an enzyme immunoassay (EIA) used to quantitatively measure the amount of the pentameric complex, wherein the pentameric complex comprises five viral proteins. A further subembodiment of the invention is wherein the five viral proteins comprise gH, gL, UL128, UL130, and UL131, which are contained in CMV drug product, drug substance, and additional process intermediary samples. The present invention comprises dosing a vaccine based on antigen content which is measured relative to a CMV containing reference standard used in this assay. The present invention comprises a method of treating a patient based on dosing a vaccine wherein the antigen content is measured relative to a CMV containing reference standard used in this assay.

A method of decreasing the likelihood of an infection by CMV of a pathology associated with such an infection in a patient, wherein the patient is treated based on dosing a vaccine, wherein the antigen content is measured relative to a CMV containing reference standard used in this assay.

An embodiment of the present invention is an assay for detecting Cytomegalovirus in a sample comprising measuring and quantitating the pentameric complex comprising: i) coating an assay plate with an antigen, ii) adding a first monoclonal antibody and incubating, iii) adding a reference standard and/or vaccine sample and incubating, iv) washing the plate, v) adding a second monoclonal antibody, vi) washing the plate, vii) adding a fluorogenic substrate, viii) calculating the glycoprotein complex, and ix) quantitating the glycoprotein complex relative to a reference standard of a sample. In a further subembodiment of the present invention, the antigen is a polyclonal antibody. In a further subembodiment of the present invention, the polyclonal antibody is a goat anti-mouse IgG1 Fc polyclonal antibody. In a further subembodiment of the present invention, the fluorogenic substrate is 4MUP.

An embodiment of the present invention is a kit for detecting and quantitating the pentameric complex, wherein the kit comprises i) an ELISA plate, ii) a goat anti-mouse IgG1Fc polyclonal antibody, iii) a detection antibody, wherein the detection antibody is a human antibody, iv) a capture antibody, wherein the capture antibody is an IgG1 mouse-rabbit chimeric antibody, v) a fluorogenic substrate, vi) an assay diluent, vii) a 2X balancing buffer, viii) a storage buffer, ix) coating buffer, and x) a wash buffer.

The methods of the present invention use a replication defective CMV (rdCMV) that expresses the pentameric gH complex. Any attenuated CMV virus that expresses the pentameric gH complex can be made replication defective. In one embodiment, the attenuated CMV is AD169 that has restored gH complex expression due to a repair of a mutation in the UL131 gene.

### Definitions

Various terms relating to the methods and other aspects of the present invention are used throughout the specification and claims. Unless defined otherwise, all technical and scientific terms have used herein have the same meaning as commonly understood by one of ordinary skill in the art to which the invention pertains. Although any methods and materials similar or equivalent to those described herein can be used in the practice for testing of the present invention, the preferred materials and methods are described herein.

It is also to be understood that the terminology used herein is for the purpose of describing particular embodiments only and is not intended to be limiting. As used in this specification and the appended claims, the singular forms “a”, “an” and “the” include plural referents unless the content clearly dictates otherwise.

“Antibody” or “immunoglobulin” is used broadly to refer to both antibody molecules and a variety of antibody-derived molecules and includes any member of a group of glycoproteins occurring in higher mammals that are major components of the immune system. The term “antibody” is used in the broadest sense and specifically covers monoclonal antibodies, polyclonal antibodies, antibody compositions with polyepitopic specificity, bispecific antibodies, diabodies, and single-chain molecules, as well as antibody fragments, so long as they exhibit the desired biological activity.

A “monoclonal antibody” (mAb) is an antibody obtained from a population of substantially homogeneous antibodies, i.e., the individual antibodies comprising the population are identical except for possible naturally occurring mutations that can be present in minor amounts.

Screening assays to determine binding specificity of an antibody are well known and routinely practiced in the art. For a comprehensive discussion of such assays, see Harlow et al., (Eds.), ANTIBODIES A LABORATORY MANUAL; Cold Spring Harbor Laboratory; Cold Spring Harbor, NY (1988), Chapter 6.

As used herein, the term “induce an immune response” refers to the ability of a conditional replication defective CMV to produce an immune response in a patient, preferably a mammal, more preferably a human, to which it is administered, wherein the response includes, but is not limited to, the production of elements (such as antibodies) which specifically bind, and preferably neutralize, CMV and/or cause T cell activation. A “protective immune response” is an immune response that reduces the likelihood that a patient will contract a CMV infection (including primary, recurrent and/or super-infection) and/or ameliorates at least one pathology associated with CMV infection and/or reduces the severity/length of CMV infection.

As used herein, the term “an immunologically effective amount” refers to the amount of an immunogen that can induce an immune response against CMV when administered to a patient that can protect the patient from a CMV infection (including primary, recurrent and/or super-infections) and/or ameliorate at least one pathology associated with CMV infection and/or reduce the severity/length of CMV infection in the patient. The amount should be sufficient to significantly reduce the likelihood or severity of a CMV infection. Animal models known in the art can be used to assess the protective effect of administration of immunogen. For example, immune sera or immune T cells from individuals administered the immunogen can be assayed for neutralizing capacity by antibodies or cytotoxic T cells or cytokine producing capacity by immune T cells. The assays commonly used for such evaluations include but not limited to viral neutralization assay, anti-viral antigen ELISA, interferon-gamma cytokine ELISA, interferon-gamma ELISPOT, intracellular multi-cytokine staining (ICS), and Chromium release cytotoxicity assay. Animal challenge models can also be used to determine an immunologically effective amount of immunogen.

Administration of rdCMV of the invention to a patient elicits an immune response to CMV, preferably a protective immune response, that can treat and/or decrease the likelihood of an infection by CMV or pathology associated with such an infection in a patient. The immune response is, at least in part, to the pentameric gH complex.

A rdCMV described herein can be formulated and administered to a patient using the guidance provided herein along with techniques well known in the art. Guidelines for pharmaceutical administration in general are provided in, for example, Vaccines Eds. Plotkin and Orenstein, W.B. Sanders Company, 1999; Remington’s Pharmaceutical Sciences 20^{th} Edition, Ed. Gennaro, Mack Publishing, 2000; and Modem Pharmaceutics 2^{nd} Edition, Eds. Banker and Rhodes, Marcel Dekker, Inc., 1990.

Vaccines can be administered by different routes such as subcutaneous, intramuscular, intravenous, mucosal, parenteral, transdermal or intradermal. Subcutaneous and intramuscular administration can be performed using, for example, needles or jet-injectors. In an embodiment, the vaccine of the invention is administered intramuscularly. Transdermal or intradermal delivery can be accomplished through intradermal syringe needle injection or enabling devices such as micron-needles or micron array patches.

The compositions described herein may be administered in a manner compatible with the dosage formulation, and in such amount as is immunogenically-effective to treat and/or reduce the likelihood of CMV infection (including primary, recurrent and/or super). The dose administered to a patient, in the context of the present invention, should be sufficient to effect a beneficial response in a patient over time such as a reduction in the level of CMV infection, ameliorating the symptoms of disease associated with CMV infection and/or shortening the length and/or severity of CMV infection, or to reduce the likelihood of infection by CMV (including primary, recurrent and/or super).

Suitable dosing regimens may be readily determined by those of skill in the art and are preferably determined considering factors well known in the art including age, weight, sex and medical condition of the patient; the route of administration; the desired effect; and the particular composition employed. In determining the effective amount of the rdCMV to be administered in the treatment or prophylaxis against CMV, the physician may evaluate circulating plasma levels of virus, progression of disease, and/or the production of anti-CMV antibodies. The dose for a vaccine composition consists of the range of 10³ to 10¹² plaque forming units (pfu). In different embodiments, the dosage range is from 10⁴ to 10¹⁰ pfu, 10⁵ to 10⁹ pfu, 10⁶ to 10⁸ pfu, or any dose within these stated ranges. When more than one vaccine is to be administered (i.e., in combination vaccines), the amount of each vaccine agent is within their described ranges.

The vaccine composition can be administered in a single dose or a multi-dose format. Vaccines can be prepared with adjuvant hours or days prior to administrations, subject to identification of stabilizing buffer(s) and suitable adjuvant composition. Vaccines can be administrated in volumes commonly practiced, ranging from 0.1 mL to 0.5 mL.

The timing of doses depends upon factors well known in the art. After the initial administration one or more additional doses may be administered to maintain and/or boost antibody titers and T cell immunity. Additional boosts may be required to sustain the protective levels of immune responses, reflected in antibody titers and T cell immunity such as ELISPOT. The levels of such immune responses are subject of clinical investigations.

For combination vaccinations, each of the immunogens can be administered together in one composition or separately in different compositions. A rdCMV described herein is administered concurrently with one or more desired immunogens. The term “concurrently” is not limited to the administration of the therapeutic agents at exactly the same time, but rather it is meant that the rdCMV described herein and the other desired immunogen(s) are administered to a subject in a sequence and within a time interval such that the they can act together to provide an increased benefit than if they were administered otherwise. For example, each therapeutic agent may be administered at the same time or sequentially in any order at different points in time; however, if not administered at the same time, they should be administered sufficiently close in time to provide the desired therapeutic effect. Each therapeutic agent can be administered separately, in any appropriate form and by any suitable route.

A “patient” refers to a mammal capable of being infected with CMV. In a preferred embodiment, the patient is a human. A patient can be treated prophylactically or therapeutically. Prophylactic treatment provides sufficient protective immunity to reduce the likelihood or severity of a CMV infection, including primary infections, recurrent infections (i.e., those resulting from reactivation of latent CMV) and super-infections (i.e., those resulting from an infection with a different stain of CMV than previously experienced by the patient). Therapeutic treatment can be performed to reduce the severity of a CMV infection or decrease the likelihood/severity of a recurrent or super-infection.

Treatment can be performed using a pharmaceutical composition comprising a rdCMV as described herein. Pharmaceutical compositions can be administered to the general population, especially to those persons at an increased risk of CMV infection (either primary, recurrent or super) or for whom CMV infection would be particularly problematic (such as immunocompromised individuals, transplant patients or pregnant women). In one embodiment, females of childbearing age, especially early adolescent females, are vaccinated to decrease the likelihood of CMV infection (either primary, recurrent or super) during pregnancy.

Those in need of treatment include those already with an infection, as well as those prone to have an infection or in which a reduction in the likelihood of infection is desired. Treatment can ameliorate the symptoms of disease associated with CMV infection and/or shorten the length and/or severity of CMV infection, including infection due to reactivation of latent CMV.

Persons with an increased risk of CMV infection (either primary, recurrent or super) include patients with weakened immunity or patients facing therapy leading to a weakened immunity (e.g., undergoing chemotherapy or radiation therapy for cancer or taking immunosuppressive drugs). As used herein, “weakened immunity” refers to an immune system that is less capable of battling infections because of an immune response that is not properly functioning or is not functioning at the level of a normal healthy adult. Examples of patients with weakened immunity are patients that are infants, young children, elderly, pregnant or a patient with a disease that affects the function of the immune system such as HIV infection or AIDS.

The recombinant virus to be used in the method of the invention also displays an immunogenic pentameric gH complex on its virion.

As used herein, the term “conditional replication defective virus” refers to virus particles that can replicate in a certain environment but not others. In preferred embodiments, a virus is made a conditional replication defective virus by destabilization of one or more proteins essential for viral replication. The nucleic acids encoding the wild type, non-destabilized essential proteins are no longer present in the conditional replication defective virus. Under conditions where the one or more essential proteins are destabilized, viral replication is decreased by preferably greater than 50%, 75%, 90%. 95%, 99%, or 100% as compared to a virus with no destabilized essential proteins. However, under conditions that stabilize the destabilized essential proteins, viral replication can occur at preferably at least 75%, 80%, 90%, 95%, 99% or 100% of the amount of replication of a CMV that does not contain a destabilized essential protein. In more preferred embodiments, one or more essential proteins are destabilized by fusion with a destabilizing protein such as FKBP or a derivative thereof. Such fusion proteins can be stabilized by the presence of a stabilizing agent such as Shield-1. As used herein, the term “rdCMV” refers to a conditional replication defective Cytomegalovirus.

As used herein, the terms “fused” or “fusion protein” refer to two polypeptides arranged in-frame as part of the same contiguous sequence of amino acids. Fusion can be direct such there are no additional amino acid residues between the polypeptides or indirect such that there is a small amino acid linker to improve performance or add functionality. In preferred embodiments, the fusion is direct.

As used herein, the terms “pentameric gH complex” or “gH complex” refer to a complex of five viral proteins on the surface of the CMV virion. The complex is made up of proteins encoded by UL128, UL130, and UL131 assembled onto a gH/gL scaffold (Wang and Shenk, 2005 Proc Natl Acad Sci USA. 102:1815; Ryckman et al, 2008 J. Virol. 82:60). The sequences of the complex proteins from CMV strain AD169 are shown at GenBank Accession Nos. NP_783797.1 (UL128), NP_040067 (UL130), CAA35294.1 (UL131), NP_040009 (gH, also known as UL75) and NP_783793 (gL, also known as UL115). Some attenuated CMV strains have one or more mutations in UL131 such that the protein is not expressed and therefore the gH complex is not formed. In such cases, UL131 should be repaired (using methods such as those in Wang and Shenk, 2005 J. Virol. 79:10330) such that the gH complex is expressed in the rdCMV of the invention. The viruses of the present invention express the five viral proteins that make up the pentameric gH complex and assemble the pentameric gH complex on the viral envelope.

The five viral proteins, gH, gL, UL128, UL130, and UL131 can also be represented as gH, gL, UL128, UL130, and UL131A or gH/gL and UL128-131.

As used herein, the term “essential protein” refers to a viral protein that is needed for viral replication in vivo and in tissue culture. Examples of essential proteins in CMV include, but are not limited to, IE1/2, UL37×1, UL44, UL51, UL52, UL53, UL56, UL77, UL79, UL84, UL87 and UL105.

As used herein, the term “destabilized essential protein” refers to an essential protein that is expressed and performs its function in viral replication and is degraded in the absence of a stabilizing agent. In preferred embodiments, the essential protein is fused to a destabilizing protein such as FKBP or a derivative thereof. Under normal growth conditions (i.e., without a stabilizing agent present) the fusion protein is expressed but degraded by host cell machinery. The degradation does not allow the essential protein to function in viral replication thus the essential protein is functionally knocked out. Under conditions where a stabilizing agent such as Shield-1, is present the fusion protein is stabilized and can perform its function at a level that can sustain viral replication that is preferably at least 75%, 80%, 90%, 95%, 99% or 100% of the amount of replication of a CMV that does not contain a destabilized essential protein.

“V160” is engineered as a replication-defective CMV, and its replication in culture is controlled by a synthetic chemical. V160 cannot replicate in humans but it maintains all virologic properties for presentation of viral antigens, including gH, gL, UL128, UL130, and UL131 pentameric complex, important for potent neutralizing antibodies (NABs).

“Drug product” or “DP” comprises a finished dosage form, for example, a tablet, capsule or solution that contains an active pharmaceutical ingredient, generally, but not necessarily, in association with inactive ingredients.

“Drug substance” or “DS”, also known as “active pharmaceutical ingredient”, comprises any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure or function of the body.

“Process intermediary” or “Process Intermediates” comprises such samples of intermediate steps throughout the purification process, for example, but not limited to, Anion Exchange Product (AEXP), Capto Core Product (COREP), Bioburden Reduced Filtrated Product (BRFP), and Sterile Filtered Product (SFP).

“ELISA” (enzyme-linked immunosorbent assay) is a plate-based assay technique designed for detecting and quantifying substances such as peptides, proteins, antibodies and hormones. Other names, such as enzyme immunoassay (EIA), are also used to describe the same technology. In an ELISA, an antigen must be immobilized on a solid surface and then complexed with an antibody that is linked to an enzyme. Detection is accomplished by assessing the conjugated enzyme activity via incubation with a substrate to produce a measurable product. The most crucial element of the detection strategy is a highly specific antibody-antigen interaction.

“Reference standard” is a highly characterized, standardized, and validated reference material, which is used to validate purity of substances for pharmaceutical use and medicinal products.

### Abbreviations

Abbreviations used are those conventional in the art of the following.

| | |
|---|---|
| 4MUP | 4-Methylumbelliferyl phosphate |
| BSA | Bovine Serum Albumin |
| CMV | Cytomegalovirus |
| DLS | dynamic light scattering |
| EC50 | half maximal effective concentration |
| ED50 | Effective Dose 50 |
| ELISA | Enzyme Linked Immunosorbent Assay |
| ELISPOT | enzyme-linked immune absorbent spot |
| IC50 | half maximal inhibitory concentration |
| IVRP | In Vitro Relative Potency |
| mcg | micrograms |
| mL | milliliters |
| mM | millimolar |
| NaCl | Sodium Chloride |
| nM | nanomolar |
| ORF | open reading frame |
| P188 | Poloxamer 188 |
| PS20 | Polyoxyethylene (20) sorbitan monolaurate (Polysorbate 20 or Tween 20) |
| PS80 | Polysorbate 80 |
| RSD | Relative standard deviation |
| TBS | tert-Butyldimethylsilyl Ethers |
| vol | volume |
| U | units |

The present invention is directed towards a sandwich Enzyme Linked Immunosorbent Assay (ELISA) used for measuring V160 dose through quantitation of Cytomegalovirus (CMV) pentamer antigen. It is an enzyme linked immunoassay (EIA) used to quantitatively measure the amount of the pentameric complex (five viral proteins - gH, gL, UL128, UL130, and UL131) contained in CMV drug substance, drug product, and additional process intermediary samples. The present invention expresses the concentration of the key glycoprotein complex, which ensures an appropriate concentration of this critical glycoprotein complex is present in the vaccine. The vaccine dose is based on antigen content, which is measured relative to a V160 containing reference standard used in this assay.

The recombinant virus to be used in the method of the invention displays an immunogenic pentameric gH complex on its virion. This pentameric gH complex comprises gH, gL, UL128, UL130, and UL131. Neutralizing antibodies directed towards this pentamer are a key component of natural immunity to CMV. The CMV vaccine uses a replication defective CMV (rdCMV) that expresses the pentameric gH complex. The attenuated CMV is AD169 that has restored gH complex expression due to a repair of a mutation in the UL131 gene.

The Sandwich ELISA that quantifies the pentamer antigen ensures consistent level of antigenicity and pentameric content per dose.

The present invention which comprises an ELISA that quantifies specifically the pentameric complex. The two mAbs used in the ELISA assay have been characterized regarding the epitope recognized by the complementary determining region (CDR) of the antibody. Through mapping, it is known the two mAbs bind different epitopes on the pentameric complex of the V160 vaccine. The present invention is improved over the quantification method previously used as the previous method detected a pAb (polyclonal antibody), which was produced by different human beings and is a mixture of antibodies that recognize different proteins in the V160 vaccine (pentameric and non-pentameric proteins). The previously used method measured a response, but not specifically from the pentameric complex. The present invention comprises an ELISA that directly measures the pentameric complex, which comprises five viral proteins – gH, gL, UL128, UL130, and UL131, wherein the ELISA expresses the concentration of the key glycoprotein complex (pentameric complex), which ensures an appropriate concentration of this critical glycoprotein complex is present in the vaccine, and therefore ensure adequate dosing of CMV to patients in need thereof.

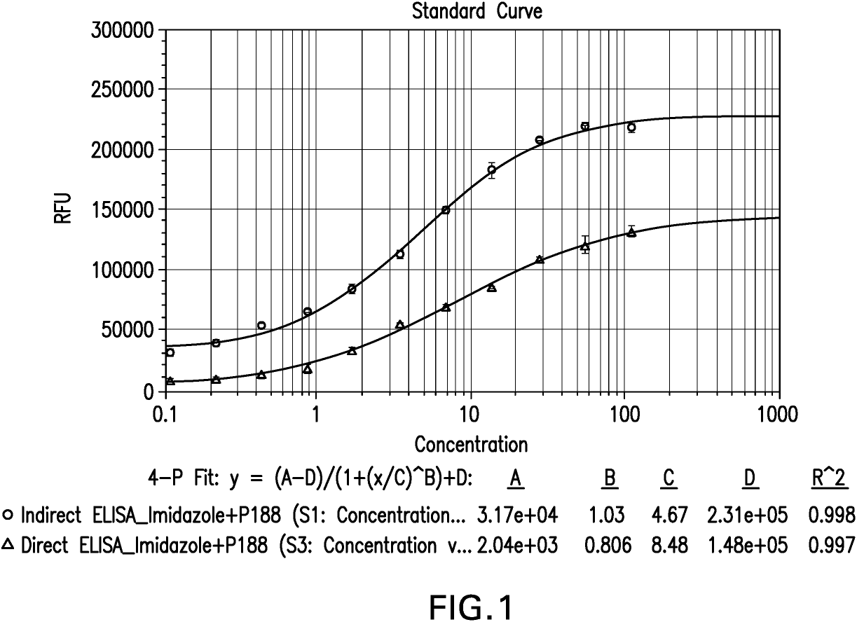

A comparison of an indirect and direct ELISA method is shown in FIG. 1. FIG. 1 is a line graph depicting the relative fluorescence units versus concentration comparing a direct and indirect ELISA. The Indirect format has a higher degree of sensitivity and is more sensitive than the direct format as indicated by the two-fold decrease in EC50.

### EXAMPLES

### Comparative Example: ELISA Assay Procedure for Determining Dosage Indirectly Quantifying the Amount of Pentameric Complex, in Addition to other Glycoproteins

### Materials

1. Assay Diluent - 1X TBS (25 mM Tris, 0.15 M NaCl, pH 7.2), 0.5% w/v BSA, 0.05% w/v P188 pH 7.2 (Merck)
2. 2X Balancing Buffer - 2X TBS (50 mM Tris, 0.3 M NaCl, pH 7.2), 1% w/v BSA, 0.1% w/v P188, pH 7.0 (Merck)
3. Storage Buffer - 25 mM Histidine, 75 mM NaCl, 9% w/v Sucrose, pH 7.0 (Merck)
4. Coating Buffer - 1X TBS (25 mM Tris, 0.15 M NaCl, pH 7.2)
5. Wash Buffer - 1X TBS (25 mM Tris, 0.15 M NaCl, pH 7.2)

### Reagents

1. Goat anti-mouse IgG1Fc polyclonal antibody (Jackson ImmunoResearch Laboratories, Cat 115-005-205) - 4 mcg/mL
2. 57.4C rabbit/mouse chimeric mAb (IgG1) (GenScript) - 4 mcg/mL
3. Cytogam® (CSL Behring Cat NDC 44206-531-11) - 48 mcg/mL
4. Goat anti-human IgG (Fc) AP (Jackson ImmunoReseach Laboratories Catalog No. 109-055-098) - 1:3000
5. 4-Methylumbelliferyl phosphate (4-MUP) (Virolab, Catalog No. X-Phos100)

### Plates

1. Nunc Maxisorp Black, 96 well, (ThermoScientific Cat 12-566-44)

The indirect Sandwich ELISA for measuring V160 content is an enzyme immunoassay (EIA) used to quantitatively measure the indirect amount of pentameric complex in addition to other glycoproteins contained in V160 drug substance, drug product, or additional process intermediary samples. The vaccine dose is based on antigen content, which is measured relative to a V160 containing reference standard using this assay.

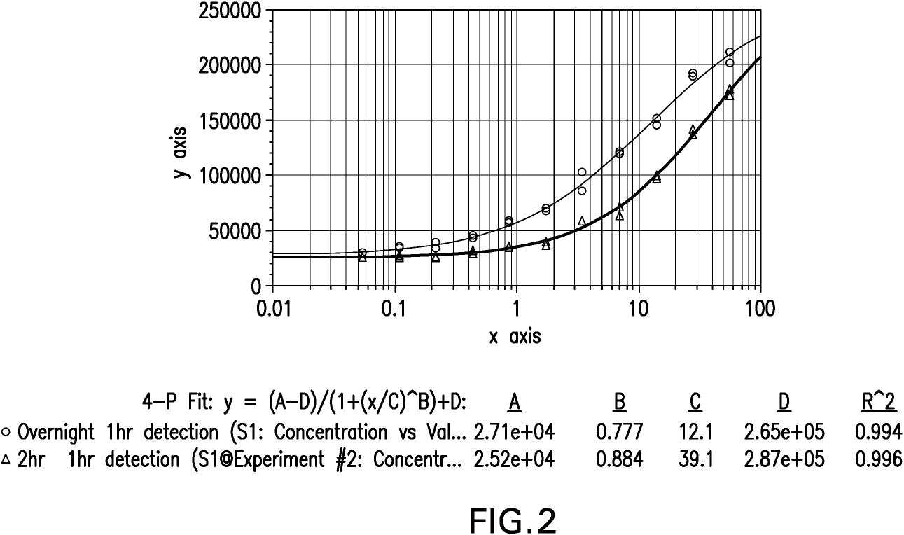

The assay uses an indirect capture format. Plates are coated with a goat anti-mouse IgG1Fc polyclonal antibody (4 mcg/mL), which is used to capture purified 57.4C mouse chimeric IgG1 monoclonal antibody (mAb) (4 mcg/mL), a neutralizing mAb (57.4C rabbit/mouse chimeric mAb) that binds CMV particles in the sample and reference standard. Overnight incubation was selected based on the increase in sensitivity (see FIG. 2). FIG. 2 is a line graph depicting the relative fluorescence units versus concentration for overnight sample incubation versus 2-hour sample incubation. Overnight sample incubation results in a more sensitive assay as indicated by the three-fold decrease in EC50. After overnight incubation of the capture mAb, both the vaccine sample (V160 drug substance, drug product, or additional process intermediary) and the reference standard are incubated for 2 hours at room temperature in a Tris-buffered saline solution containing 0.5% Bovine Serum Albumin (BSA) and 0.05% P188 in the antibody coated microtiter plate. CytoGam®, Cytomegalovirus Immune Globulin (Human) (48 mcg/mL), an Immunoglobulin G (IgG) containing a standardized amount of antibody to Cytomegalovirus (CMV), is then added to the microtiter plate. An alkaline-phosphatase conjugated anti-Human IgGFc specific antibody (1:3000) is used to detect the final complex. A fluorogenic substrate (4-MUP) is added to the plate and signal develops in proportion to the amount of CMV antigen bound to the plate. The resulting relative fluorescence units (RFU), which are related to the extent of the reaction, are read on a microtiter plate reader at an excitation wavelength of 360±5 nm and an emission wavelength of 450±5 nm.

A relative potency value is calculated for each sample by comparison to the reference standard which is used to calculate the antigen content (units/mL). For information, the specific activity of the sample may be calculated by dividing the antigen content by the sample mass as determined using the Bradford assay or another mass assay.

While the capture mAb for this ELISA is specific for an epitope on the pentameric complex, the detection reagent (Cytogam®) is a polyclonal preparation from multiple human sources and is not specific for the pentameric complex. The Cytogam® reagent may recognize multiple glycoproteins on the surface of the virus.

### Example 1: Selecting Assay Conditions

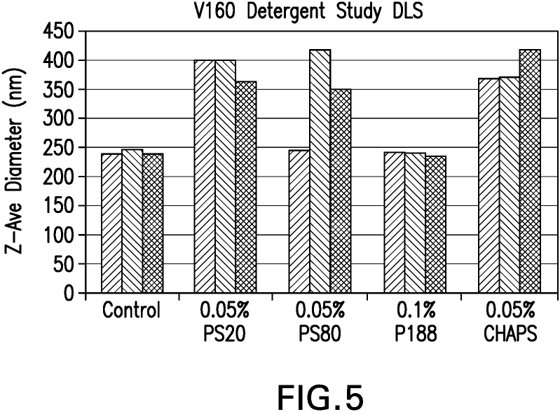

Example 1 used Tween 20 to prevent non-specific adsorption of protein to surfaces. Various techniques were implemented to evaluate the V160 process consistency (i.e., DLS, Disk Centrifugation and flow cytometry). During the course of evaluating V160 process material, an observation was made that V160 material exposed to Tween 20 in an assay buffer matrix resulted in a particle profile shift (measured by flow cytometry) when compared to the general V160 storage buffer (HNS - Histidine, NaCl and Sucrose). The mechanism of the Tween 20 effect is not fully understood but it is expected to be related to the disruption of the viral membrane by the detergent and subsequent loss of tegument layer in viral particles and thus reduction of their size. Tween 20 is a standard detergent/surfactant used in most ELISA buffers to decrease assay background and prevent adsorption of proteins to different surfaces. The assay buffer used for the ELISA as represented in the previous comparative example initially contained 0.05% Tween 20. Further detergents/surfactants were evaluated for use in the V160 ELISA in response to the effect of Tween 20 on the V160 particle profile (see FIG. 5).

The majority of detergents tested resulted in an increase of average particle size for either the drug substance, drug product, or both, presumably as a result of particle aggregation after membrane disruption by the detergent. Numerous detergents (anionic, cationic, zwitterionic, and non-ionic) were evaluated for use in the V160 ELISA. The optimal detergent would maintain a particle profile similar to the HNS (Histidine, NaCl and Sucrose) storage buffer control and the ability to prevent adsorption to plastic. The majority of detergents tested (at CMC - Critical Micelle Concentration) resulted in a change in particle profile for either the drug substance, drug product or both. P188 (nonionic linear copolymer) was studied and resulted in a particle profile identical to HNS (Control) and prevented adsorption to plastic. P188 was therefore selected for use in the assay buffer. P188 concentrations ranging from 0.03 to 8.1% in combination with V160 were evaluated using flow cytometry, Disk Centrifugation and Adsorption (measured using Colloidal Gold Staining). Various concentrations of P188 within this range were evaluated in the ELISA for optimal performance. P188 at 0.05% was selected for optimal use.

In summary, P188 did not affect particle size and distribution measured by DLS and flow cytometry but helped to minimize adsorption of the virus. Therefore, P188 was used in the minimize absorption of V160 to pipet tips, tubes, among other additional assay materials.

FIG. 5 is a bar chart representing the comparison of different detergents evaluated for use in the ELISA assay. The majority of detergents tested (at CMC - Critical Micelle Concentration) resulted in an increase of average particle size for either the drug substance, drug product or both, presumably as a result of particle aggregation after membrane disruption by the detergent. P188 (nonionic linear copolymer) was studied and resulted in a particle profile identical to HNS (Control) and prevented adsorption to plastic.

The Z-ave diameter is obtained by Dynamic Light Scattering (DLS) which can determine particle size by measuring Brownian motion by monitoring the random change in the intensity of light scattered from a suspension or solution.
0.05% PS20 represents polysorbate 20 (Tween 20) at a 0.05% concentration.
0.05% PS80 represents polysorbate 80 at a 0.05% concentration.
0.1%P188 represents Poloxamer 188 at 0.1% concentration.
0.05% CHAPS represents a surfactant or detergent, 3-[(3-cholamidopropyl)dimethylammonio]-1-propanesulfonate, at a 0.05% concentration.

The first bar in FIG. 5 for each Detergent (ex. Control, 0.05% PS20, 0.05% PS80 etc) is drug substance, the second bar is drug product and the third is both results combined.

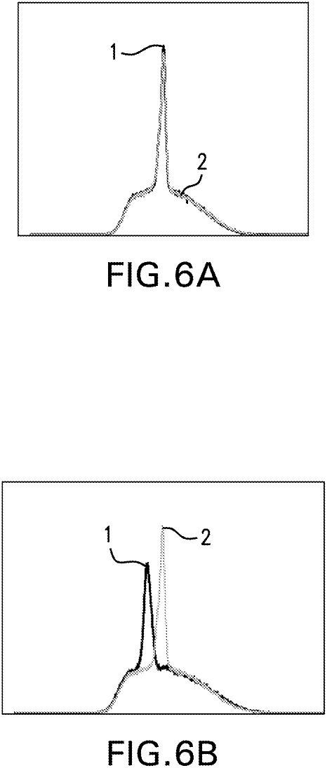

FIGS. 6A and 6B are particle profiles generated using flow cytometry representing the comparison of different detergents evaluated for use in the ELISA assay. The majority of detergents tested, including Tween 20 (at CMC - Critical Micelle Concentration) resulted in a change in particle profile for either the drug substance, drug product or both, as shown in FIG. 6B. P188 (nonionic linear copolymer) was studied and resulted in a particle profile identical to HNS (Control) and prevented adsorption to plastic as shown by the two overlaying lines in the particle profile. FIG. 6B shows an earlier PS20 particle profile than that of P188 buffer.

In FIG. 6A: The viral particles are found in the peak (“1”), whereas the dense bodies are found in the lower part of the peak (“2”). The peak is represented by two lines, P188 buffer and HNS, but both are identical and indistinguishable from one another.

In FIG. 6B: The PS20 buffer peak is represented by peak 1 (“1”) and the P188 buffer is represented by peak 2 (“2”).

### Example 2: ELISA Assay Procedure for Determining Dosage by Quantitatively Measuring the Amount of Pentameric Complex, without Measuring Additional Glycoproteins

### Materials

1. Assay Diluent - 10 mM Imidazole, 150 mM NaCl, 0.5% BSA, 0.05% P188 pH 7.0 (Merck)
2. 2X Balancing Buffer - 20 mM Imidazole, 225 mM NaCl, 1% BSA, 0.1% P188, pH 7.0 (Merck)
3. Storage Buffer - 25 mM Histidine, 75 mM NaCl, 9% w/v Sucrose, pH 7.0 (Merck)
4. Coating Buffer - 1X TBS (25 mM Tris, 0.15 M NaCl, pH 7.2)
5. Wash Buffer - 1X TBS (25 mM Tris, 0.15 M NaCl, pH 7.2)
6. 30% P188 solution in water

### Reagents

1. Goat anti-mouse IgG1Fc polyclonal antibody (Jackson ImmunoResearch Laboratories, Cat 115-005-205) - 3 mcg/mL
2. 57.4C rabbit/mouse chimeric mAb (IgG1) (GenScript) - 1.5 mcg/mL
3. D1-103 human mAb (GenScript) - 0.5 mcg/mL
4. Donkey anti-human IgG (H+L) AP (Jackson ImmunoReseach Laboratories Cat 709-055-149) - 1:1000
5. 4MUP (Virolab, Cat X-Phos100)
6. 15% P188

### Plates

1. Half Area, Black Microtiter plate, 96 well, (Griener BioOne Cat 675077)

The indirect sandwich enzyme linked immunosorbent assay (ELISA) was used to quantitatively measure the amount of pentameric complex, without measuring additional glycoproteins, contained in V160 drug substance, drug product samples, or additional process intermediary samples. Without measuring the multiple glycoproteins on the surface of the virus, this indirect sandwich ELISA targets the specific pentameric complex.

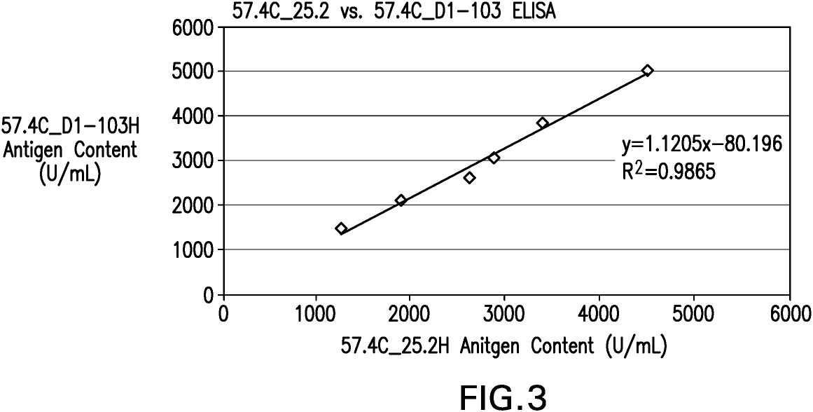

The assay was used to measure V160 dose and potency for release and stability. Test results are reported relative to a V160 containing reference standard which is stored at -70 ±10° C. Two monoclonal antibodies directed against different epitopes on the pentameric complex are used to specifically quantitate the glycoprotein complex. The antibodies used include 57.4C, an IgG1 mouse-rabbit chimeric antibody that has been shown to be neutralizing against CMV, and D1-103H, a human antibody isolated from patients with a naturally occurring CMV infection that has also been shown to be neutralizing (see FIG. 3). FIG. 3 is a line graph showing either assay, 57.4C_25.2H or 57.4C_D1-103H (also referred to as 57.4_25.2 or 57.4_D1-103), is a suitable pair for measuring the pentameric complex. The comparison of results for drug substance lots from both assays results in an R² of 0.9865, representing no statistical significance between the two methods.

Half area 96 well plates were coated with a final concentration of 3 mcg/mL of goat anti-mouse IgG1Fc polyclonal antibody, for approximately 1 hour. The plates were blocked for 30-60 minutes, followed by incubation of 1.5 mcg/mL 57.4C mouse-rabbit chimeric capture antibody. The reference standard (with P188 spike) and vaccine sample (with P188 spike) were added to the plate and subsequently serially diluted across the plate. The samples and reference standard were incubated overnight at ambient temperature. The plates were washed, and 0.5 mcg/mL of D1-103H human mAb was added to the plate and incubated for 1 hour. An alkaline phosphatase conjugated Donkey anti-Human IgG (H+L) specific antibody (1:1000) was used to detect the final complex.

After incubation, a final wash step was performed and a fluorogenic substrate (4MUP) was added to the plate and signal develops in proportion to the amount of CMV antigen bound to the plate. The resulting relative fluorescence units (RFU), which are related to the extent of the reaction, were read on a microtiter plate reader at an excitation wavelength of 360 ± 5 nm and an emission wavelength of 450 ± 5 nm.

All steps, except the blocking and 4MUP incubations, were incubated at room temperature with shaking. The 4MUP incubation was performed at 23° C.

A relative potency value was calculated for each sample by comparison to the reference standard which was used to calculate the antigen content (units/mL). Data analysis was performed using a 4-parameter logistic function and parallel line analysis. The antigen content was determined by taking the ratio of the sample ED50 relative to the reference ED50 and multiplying by the antigen content of the reference standard. The assay range in which the antigen content is accurately measured is 800-25 U/mL.

An example of sample preparation as described below:
The reference standard at 200 U/mL was spiked to a final concentration of 1.5% P188 and 100 mcg/mL Acridine Orange
The sample to be tested, the neat DP (drug product in its original form, as in undiluted) was spiked to a final concentration of 1.5% P188 and 100 mcg/mL Acridine Orange
The neat DS was diluted 1:5 and spiked to a final concentration of 1.5% P188 and 100 mcg/mL Acridine Orange.

### Example 3: D300 Digital Dispenser for Use in Performing the ELISA as Represented in Example 2

### Materials

1. Assay Diluent - 10 mM Imidazole, 150 mM NaCl, 0.5% BSA, 0.05% P188 pH 7.0 (Merck)
2. Storage Buffer - 25 mM Histidine, 75 mM NaCl, 9% w/v Sucrose, pH 7.0 (Merck)
3. Coating Buffer - 1X TBS (25 mM Tris, 0.15 M NaCl, pH 7.2)
4. Wash Buffer - 1X TBS (25 mM Tris, 0.15 M NaCl, pH 7.2)
5. 30% P188 solution in water

### Reagents

1. Goat anti-mouse IgG1Fc polyclonal antibody (Jackson ImmunoResearch Laboratories, Cat 115-005-205) - 3 mcg/mL
2. 57.4C rabbit/mouse chimeric mAb (IgG1) (GenScript) - 1.5 mcg/mL
3. D1-103H human mAb (GenScript) - 0.5 mcg/mL
4. Donkey anti-human IgG (H+L) AP (Abcam Cat ab) - 1:1000
5. 4MUP (Virolab, Cat X-Phos100)
6. 15% P188 / 1000 mcg/mL Acridine Orange
Note: Acridine Orange is a fluorescent dye used as an internal control to measure dilution bias

### Plates

1. Half Area, Black Microtiter plate, 96 well, (Griener BioOne Cat 675077)

The HP D300 Digital Dispenser is an automated benchtop instrument that uses HP’s Direct Digital Dispensing technology to enable rapid delivery of picoliter to microliter sample volumes to create dose response curves (IC50/EC50) for biological material. The HP D300 digital dispense enables the elimination of serial dilutions, and easily dispenses any volume in any well for a broad array of low-volume dispensing applications.

Reference samples were typically plated and titrated in individual rows of a 96 well plate. Plate positional effects may result in non-uniform raw signal measurements across the 96 well plate. Small positional effects may result in variable assay data. The D300 in combination with positional randomization across the plate is being used as a mitigation strategy to decrease or eliminate positional effects. A block randomization scheme is employed to minimize errors at the inflection point of the dose response curve, thereby reducing relative potency bias.

For sample preparation details, refer to Example 2.

The ELISA was performed as described above in Example 2 with the following changes:
The samples were loaded directly onto a T8+ cassette on the D300.
The D300 was programmed to dispense the sample in a randomized manner

The D300 provided additional benefits including; increasing accuracy, no or limited sample serial dilution was necessary, and the sample position was randomized therefore reducing the plate positional effects.

The use of the D300 resulted in decreased variability for drug substance and drug product results (RSD approximately +/- 5%) within a plate and across plates with in a run.

The manual ELISA (as represented in Example 1 and 2) historically adds sample to Column 1 of the 96 well plate and serially dilutes the sample across Columns 2-11. The 96 well plate typically was prone to edge effects, therefore samples on the edge of the plates (1^{st} or last row, or 1^{st} or last column of the plate) potentially were affected and may had led to lower or higher RFU values relative to the duplicate results in inner rows/ columns of the plate. The D300 allows the user to program the software to distribute the sample in a block randomization format. Instead of performing serial dilutions of a sample across the 96 well plate (Column 1-11), each sample was distributed at the requested concentration in each well and the upper curve points and those around the EC50 were plated in the center of the plate, to minimize effects of the outer rows and columns of the plate. The lower curve points closest to the background signal were randomized and plated in the outer columns and rows.

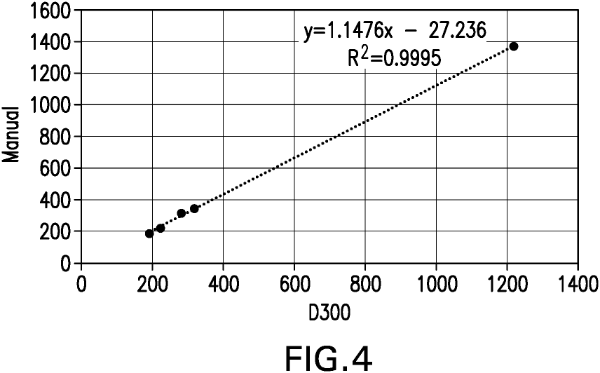

The manual ELISA was compared against the D300 Digital Dispense in FIG. 4 and there was no statistically significant difference in the antigen content result between the two ELISA testing methods. FIG. 4 is a line graph depicting the comparison of antigen content results for Drug Product and Drug substance matrices using the Manual ELISA vs. the D300 Digital Dispenser ELISA. The comparison of results for the matrices from both the manual and D300 assay results in an R² of 0.9995, representing no statistical significance between the two methods.

Three samples were tested 3 times each across 3 ELISA plates. The %RSD for each sample tested in the Randomized D300 ELISA was <4%, while the %RSD for the three samples tested in the manual ELISA was 6-12%. Randomization helps reduce the plate effects and typically results in better precision (within plate, plate to plate and day to day).

### Example 4: ELISA Assay Procedure for Determining Dosage by Quantitatively Measuring the Amount of Pentameric Complex, Without Measuring Additional Glycoproteins with the Use of Detection Monoclonal Antibody 252H Human mAb

Example 4 was performed with the same method as Example 2 with a change in the detection monoclonal antibody to 25.2H Human mAb and the concentration used.

### Reagents

1. Goat anti-mouse IgG1Fc polyclonal antibody - (Jackson ImmunoResearch Laboratories, Cat 115-005-205) - 4 mcg/mL
2. 57.4C rabbit/mouse chimeric mAb (IgG1) (GenScript) - 2 mcg/mL
3. 25.2H human mAb (GenScript) - 2 mcg/mL
4. Donkey anti-human IgG (H+L) AP (Jackson ImmunoResearch Laboratories Cat 709-055-149) - 1:1000
5. 4MUP (Virolab, Cat X-Phos100)
6. 15% P188

What is claimed is:

1. A method of detecting the presence of Cytomegalovirus (CMV) in a sample, comprising measuring the amount of pentameric complex in a sample using an indirect sandwich enzyme linked immunosorbent assay (ELISA), and quantifying the amount against a reference sample with a known amount of CMV, wherein
the sample is a drug substance sample, drug product sample, or process intermediary sample.

2. The method of claim 1, wherein detecting and quantifying the presence of Cytomegalovirus (CMV) does not detect and quantify multiple glycoproteins.

3. The method of claim 1, wherein the pentameric complex comprises gH, gL, UL128, UL130, and UL131.

4. The method of claim 3, wherein the method additionally comprises P188.

5. The method of claim 1, wherein the sandwich ELISA comprises two monoclonal antibodies against different epitopes on the pentameric complex used to detect and quantitate the pentameric complex, wherein the two monoclonal antibodies comprise:
a. a capture antibody, wherein the capture antibody is an IgG1 mouse-rabbit chimeric antibody, and
b. a detection antibody selected from a human antibody isolated from patients with a naturally occurring CMV infection.

6. The method of claim 5, wherein the capture antibody is 57.4C.

7. The method of claim 5, wherein the detection antibody is selected from D1-103H or 25.2.

8. The method of claim 1, wherein the two antibodies are used to quantitate the glycoprotein complex comprising measuring the sample against relative potency of the sample is quantitated against the antigen content of a known reference standard.

9. A method for quantifying Cytomegalovirus comprising a sandwich ELISA assay measuring the amount of the pentameric complex, wherein the pentameric complex comprises gH, gL, UL128, UL130, and UL131, and wherein this amount is quantified relative to a reference standard of a sample with a known amount of CMV.

10. (canceled)

11. An assay for detecting Cytomegalovirus (CMV) in a sample comprising measuring and quantitating the glycoprotein complex comprising:
a. coating an assay plate with an antigen
b. adding a first monoclonal antibody and incubating,
c. adding a reference standard and/or vaccine sample and incubating,
d. washing the plate,
e. adding a second monoclonal antibody,
f. washing the plate,
g. adding a fluorogenic substrate,
h. calculating the glycoprotein complex, and
i. quantitating the glycoprotein complex relative to a reference standard of a sample.

12. The assay of claim 11, wherein the first monoclonal antibody is 57.4C mouse-rabbit chimeric mAb.

13. The assay of claim 11, wherein the second monoclonal antibody is D1-103H human mAb or 25.2H human mAb.

14. The assay of claim 11, wherein the antigen is a polyclonal antibody.

15. The assay of claim 11, wherein the polyclonal antibody is a goat anti-mouse IgG1Fc polyclonal antibody.

16. The assay of claim 11, wherein the fluorogenic substrate is 4MUP.

17. A kit for detecting, measuring and quantitating the glycoprotein complex, wherein the kit comprises
a. an ELISA plate,
b. a goat anti-mouse IgGlFc polyclonal antibody,
c. a detection antibody, wherein the detection antibody is a human antibody:
d. a capture antibody,
e. a fluorogenic substrate,
f. an assay diluent
g. 2X Balancing Buffer
h. a storage buffer
i. a coating buffer, and
j. a wash buffer.

18. The kit according to claim 17, wherein the detection antibody is a human antibody selected from one of the following: D1-103H or 25.2 antibody and wherein the capture antibody is 57.4, an IgGl mouse-rabbit chimeric antibody.

19. (canceled)

20. The kit according to claim 17, wherein the fluorogenic substrate is 4MUP.

21. (canceled)

22. (canceled)

23. (canceled)

24. (canceled)

25. (canceled)

26. The kit according to claim 17, wherein
a. the assay diluent comprises 10 mM Imidazole, 150 mM NaCl, 0.5% BSA, 0.05% P188 pH 7.0,
b. the 2X balancing buffer comprises 20 mM Imidazole, 225 mM NaCl, 1% BSA, 0.1% P188, pH 7.0,
c. the storage buffer comprises 25 mM Histidine, 75 mM NaCl, 9% w/v Sucrose, pH 7.0,
d. the 2X balancing buffer comprises 20 mM Imidazole, 225 mM NaCl, 1% BSA, 0.1% P188, pH 7.0,
e. the storage buffer comprises 25 mM Histidine, 75 mM NaCl, 9% w/v Sucrose, pH 7.0,
f. the coating buffer comprises 1X TBS (25 mM Tris, 0.15 M NaCl, pH 7.2), and
g. the wash buffer comprises 1X TBS (25 mM Tris, 0.15 M NaCl, pH 7.2).
